(19) United States
(12) Patent Application Publication
(10) Pub. No.: US 2026/0240905 A1
(43) Pub. Date: Aug. 20, 2026

(54) ASCORBIC ACID AND FERROUS IRON COMPOSITIONS WITH ADIPOCYTE-LOWERING EFFECT

(71) Applicant: Farmacia Metalla S.R.L., Milano (IT)

(72) Inventor: Marco Maria METALLA, Milano (IT)

(21) Appl. No.: 19/161,526

(22) PCT Filed: Mar. 1, 2024
(86) PCT No.: PCT/EP2024/055373

§ 371 Date: Sep. 2, 2025

(30) Foreign Application Priority Data
Mar. 2, 2023 (IT) 102023000003777

(51) Int. Cl. A61K 33/26; A61K 9/00; A61K 9/08; A61K 31/167; A61K 31/375; A61K 31/445; A61K 47/02; A61K 47/18; A61K 47/26; A61P 3/00

(52) U.S. Cl. CPC A61K 33/26; A61K 9/0019; A61K 9/08; A61K 31/167; A61K 31/375; A61K 31/445; A61K 47/02; A61K 47/183; A61K 47/26; A61P 3/00

(57) ABSTRACT

The present invention relates to compositions containing ascorbic acid or a physiologically acceptable salt thereof and a physiologically acceptable salt of ferrous iron as active ingredients for the preparation of a medicament, or a medical device, or a sanitary product, useful for the reduction of the number of adipocytes in localized adiposities by a controlled lipolysis without an apoptotic effect.

The present invention relates to compositions containing ascorbic acid or a physiologically acceptable salt thereof and a physiologically acceptable salt of ferrous iron as active ingredients for the preparation of a medicament, or a medical device, or a sanitary product, useful for the reduction of the number of adipocytes in localized adiposities by a controlled lipolysis without an apoptotic effect.

### BACKGROUND OF THE INVENTION

Adipose cell, also called adipocyte or fat cell, is a connective-tissue cell specialized to synthesize and contain large globules of fat. There are two types of adipose cells: white adipose cells contain large fat droplets, only a small amount of cytoplasm, and flattened, non-centrally located nuclei; and brown adipose cells contain fat droplets of differing size, a large amount of cytoplasm, numerous mitochondria, and round, centrally located nuclei. The colour of brown adipose is attributed to its relatively high density of mitochondria and its extensive vascular supply [Encyclopaedia Britannica 2020 https://www.britannica.com/science/adipose-cell. Accessed 6 Jan. 2023]. The chief chemical constituents of adipose cell fat are triglycerides, which are esters made up of a glycerol and one or more fatty acids, such as stearic, oleic, or palmitic acids. Lipases, i.e. enzymes contained in adipose cells specialize in the hydrolysis of triglycerides in order to generate fatty acids and glycerol for physiological processes. The fat stored in these cells in part comes directly from the fats eaten and in part is synthesized within the body from fats and carbohydrates in the food and sometimes from protein. The main reservoir of fat in the body is the adipose tissue beneath the skin, called the panniculus adiposus. There are also deposits of fat between the muscles, among the intestines and in their mesentery, around the heart, and elsewhere. One function of these deposits is to act as soft elastic padding between the various organs.

Adipose tissue, which is in great part made up of adipose cells, acts as a fuel reserve and helps conserve the heat of the body. In times of significant energy expenditure (e.g., exercise) or lack of adequate energy intake (e.g., fasting), adipose cells secrete fatty acids, which can be used by muscles and other tissues as a source of energy. Fatty acids generated by brown adipose cells generally are not secreted; instead, they are used by the cells' mitochondria in order to generate heat (thermogenesis), particularly in hibernating animals and human infants.

When old adipose cells die during the programmed cell death physiological phenomenon called apoptosis they are replaced by new cells and in adult humans the number of adipose cells in the body is relatively stable. As a result, fat storage is accomplished through the expansion of existing adipose cells instead of through an increase in the number of cells. Likewise, when fat is utilized by the body, adipose cells shrink in size, as opposed to decreasing in number. The amount of fat contained in adipose cells can have significant effects on overall health. For example, too little fat can lead to reproductive dysfunction (e.g., amenorrhea in women), whereas excess fat can lead to obesity, cardiovascular disease, cancer, or diabetes.

Because brown adipose cells function primarily as energy consumers and white adipose cells function primarily as energy stores, the common origin of these cells has been debated. In general, brown and white adipose cells are not found together; they form distinct tissues. Brown adipose cells and muscle cells originate from the same precursor cells. In contrast, white adipose cells do not arise from the precursors of brown adipose cells and do not have any features of muscle cells. The discovery of the presence of brown adipose tissue in adult humans indicates that insight into the mechanisms underlying brown adipose cell development may have implications in the treatment of obesity [Encyclopaedia Britannica 2020, cited].

Excessive localized fat and body weight may weaken health, acknowledging different multiple factors such as physical, psychological, and genetic causes. The increasing demand for methods of body fat reduction is due by the fact that 13% of the world's adult population (15% of women and 11% of men) were obese in 2016 and the worldwide prevalence of obesity nearly tripled between 1975 and 2016 [WHO World Health Statistics Report 2021, https://www.who.int/news-room/fact-sheets/detail/obesity-and-overweight. Accessed 7 Jan. 2023].

Localized adiposity is the accumulation of subcutaneous adipose tissue, placed in definite anatomic areas, building up an alteration of the body silhouette. Usually, undesirable fat is situated in the abdomen, flanks, and thighs back, arms, and chest. Excessive localized fat is also an aesthetical problem: physical appearance has great value, and many patients who suffer from excessive localized fat feel weakened with a big impact on relationships with friends or partners, on the social environment, lowering self-esteem, and affects personal and professional life in general. The reduction in the calories consumed, a balanced diet and lifestyle changes must be recommended for weight loss and the reduction in risk factors. Liposuction or injection lipolysis techniques have been proposed for removing the excess of subcutaneous fat for improving the aesthetical appearance. Liposuction is a gold-standard procedure used in clinical practice [Matarasso A et al. (2013) Evidence-based medicine: liposuction. Plast Reconstr Surg 132(6): 1697-1705.14] however, it is expensive and is a surgical procedure which often needs general anaesthesia. Furthermore, it is not devoid of risk of sometime fatal accidents, such as postoperative infection, thromboembolic disease, skin irregularity, fat embolism, pulmonary oedema, lidocaine intoxication, and intraabdominal visceral lesion [Lu J et al. Infectious shock after liposuction. BMC Infect Dis. 2022 Jul. 15; 22(1): 617]. For these reasons, most studies propose particularly non-invasive techniques for the reduction of subcutaneous fat layers such as injections of deoxycholic acid [Amore R et al. (2019) Non-surgical treatment of lower eyelid fat pads with an injectable solution acid deoxycholic based. J Biol Regul Homeost Agents 33(6 Suppl. 2): 109-114], cryolipolysis [Abboud S et al. (2020) Heat shock lipolysis: radiofrequency combined with cryolipolysis for the reduction of localized subcutaneous fat. Dermatol Res Pract. 2020:4093907.], radiofrequency ablation [Pumprla J, Howorka K, Kolackova Z, Sovova E (2015) Noncontact radiofrequency-induced reduction of subcutaneous abdominal fat correlates with initial cardiovascular autonomic balance and fat tissue hormones: safety analysis. F1000Res 4:49], and high-intensity focused ultrasound (HIFU) [Park H et al. (2015) High-intensity focused ultrasound for the treatment of wrinkles and skin laxity in seven different facial areas. Ann Dermatol 27(6): 688-693].

Today, more patients demand non-invasive techniques for reducing local fat, representing now the fastest growing area of aesthetic medicine. The first technique proposed was infra-adipose injections of phosphatidylcholine and deoxycholic acid which were available for efficacious chemical lipolysis. Vitamin C (ascorbic acid) was used mixed with Klein solution (a mixture of corticosteroid, adrenaline and a local anaesthetic agent) because it increased lipolysis and could improve skin retraction where liposuction did not achieve good results [Senen D et al. (2002) Contribution of vitamin C administration for increasing lipolysis. Aesthetic Plast Surg 26(2): 123-125]. Based on the fact that adipose cells do not increase their number in the human body, killing them is considered as a well effective method to reduce localised adiposities and obesity. Nevertheless, all those methods are not devoid of intrinsic risk of toxicity of the invasive procedures or of the drugs injected by the above techniques.

A treatment for local adiposity by injection of a mixture of ascorbic acid (vitamin C) and ascorbyl-palmitate solution has been recently proposed [Scarano et al. A New Treatment for Local Adiposity with Ascorbic Acid and Ascorbyl-Palmitate Solution: Clinical and Histological Study. Aesth Plast Surg (2020) 44:1604-1612]. The authors describe their effect as the result apoptosis of adipocytes induced by their solution. Nevertheless, apoptosis is a process inducing other new cells be formed by a stimulation induced by the apoptotic cells, therefore the final outcome of such a procedure may be the regrowth of new adipocytes replacing those died by apoptosis. Moreover, the induction of apoptosis is not devoid of risk if cell clearance process is disrupted, and there is an accumulation of uncleared corpses, as uncontrolled release of pro-inflammatory cytokines by the apoptotic adipocytes may lead to local inflammatory reactions [Ravichandran K S. Beginnings of a good apoptotic meal: the find-me and eat-me signaling pathways. Immunity. 2011 Oct. 28; 35(4): 445-55].

There is therefore the need of a method for the treatment of local adiposity in a subject that is devoid of the risks of the surgical treatments and that is less invasive and toxic than the local treatments by injection known in the art.

Ascorbic acid is very well known in the art [Martindale, The complete drug reference, 33rd edition, S. C. Sweetman Ed., Pharmaceutical Press 2002]. Ascorbic acid, a water soluble vitamin, is essential for the synthesis of collagen and intercellular material. Vitamin C deficiency develops when the dietary intake is inadequate. It is rare in adults, but it may occur in infants, in alcohol addicts, or in elderly. Deficiency leads to the very well defined syndrome known as scurvy. This is characterized by capillary fragility, bleeding (especially from small blood vessels and the gums) normocytic or macrocytic anaemia, cartilage and bone lesions, and slow healing of wounds.

Ascorbic acid is used in the treatment and prevention of deficiency, and it completely reverses the symptoms and signs. It is usually given by mouth, the preferred route, in form of tablets or capsules, and has been given to children in the form of a suitable fruit juice, such as orange juice or as black currant or rose hip syrups. Water soluble salts of ascorbic acid may be administered parenterally or subcutaneously.

Therapeutically, vitamin C is also used following surgical interventions, as an adjuvant to chelating agent, to increase iron excretion, or in combination to iron compositions to improve oral absorption of iron, for the faster healing of bone fractures, and as a general tonic. Higher vitamin C doses are recommended as a preventative against catching colds and for speeding up the wound healing. Ascorbic acid is used as an antioxidant in pharmaceutical manufacturing and in the food industry.

A beneficial effect of a “megadose” ascorbic acid therapy has been claimed for an extraordinary number of conditions including asthma, atherosclerosis, cancer, psychiatric disorders, infertility and osteogenesis imperfecta, but there is little evidence of real effectiveness [Martindale 2022, cited].

Among the possible beneficial effects of ascorbic acid on obesity, it has been suggested that vitamin C may act as follows: modulate adipocyte lipolysis; regulate the glucocorticoid release from adrenal gland; inhibit glucose metabolism and leptin secretion; decrease glycosylation in obese-diabetic models; reduce the inflammatory response [Garcia-Diaz F et al. Vitamin C in the treatment and/or prevention of diabetes. J Nutr Sci Vitaminol, 2014.60:367-369].

Ferrous iron salts are typically used in medicine to provide iron supplementation in the states of iron deficiency. Elemental iron is contained in haemoglobin, the protein of the red blood cells and contributes to the transport of oxygen from the lung to the rest of the body for its oxidative metabolism. But elemental iron is also contained in myoglobin, the protein of muscles. Skeletal muscle contains 10-15% of iron in the body [Stugiewicz M et al. The influence of iron deficiency on the functioning of skeletal muscles: experimental evidence and clinical implications. Eur J Heart Fail 2016; 18:762-73]. Iron is fundamental to oxidative metabolism in skeletal muscle, both for efficient oxygen storage in myoglobin and for an optimal activity of mitochondrial enzymes. Iron is present in a larger amount in slow, ‘red’ fibres, which are common in e.g. dorsal muscles, lower extremity extensors, the diaphragm, and intercostal muscles. In muscle, iron is of a particular importance for oxygen reducing systems in order to provide efficient ATP production. Oxidative energy generation takes place in mitochondria via the mitochondrial respiratory chain composed of four complex transmembrane iron-containing enzymes [Stugiewicz M et al, cited]. It has also been postulated that iron deficiency is an important causative factor of loss of muscle oxidative capacity that significantly contributes to skeletal myopathy seen in patients with heart failure, chronic obstructive pulmonary disease and type 2 diabetes mellitus, therefore it may be considered as a co-target in a therapeutic process [Dziegala M et al, Iron deficiency as energetic insult to skeletal muscle in chronic diseases. J Cachexia Sarcopenia Muscle. 2018 October; 9(5): 802-815].

Water soluble ferrous salts are orally administered as iron supplementation in iron deficiency, but in case of poor absorption or more severe anaemic state intravenous injection is indicated.

### DESCRIPTION OF THE INVENTION

It has now been surprisingly found that compositions containing ascorbic acid or a physiologically acceptable salt thereof and a physiologically acceptable salt of ferrous iron, when injected directly into localised adiposities are useful for the reduction of the number of adipocytes in the sites of injection by a controlled lipolysis which is not mediated by any effect on programmed cell death or apoptosis. This effect is superior to the one described [Senen et al. (2002), cited] by using Vitamin C mixed with Klein solution, due to the fact that the prior art exposes the patient to the intrinsic toxicity of corticosteroid, adrenaline and local anaesthetic agent. The effect is also superior to the one disclosed by Scarano [Scarano et al. 2020, cited] as it causes a controlled lipolysis without inducing apoptosis of the adipocytes.

An embodiment of the present invention is therefore a pharmaceutical composition comprising (1) a water solution of ascorbic acid or a physiologically acceptable salt thereof and (2) a water solution of a ferrous iron salt for use as a medicament, wherein said composition is administered by injection within 10 minutes after mixing said two water solutions (1) and (2).

Preferably said composition is administered by injection within 2 to 10 minutes after mixing said two water solutions (1) and (2), more preferably within 3 to 6 minutes, even more preferably at about 3 minutes.

According to a preferred embodiment said pharmaceutical composition is used in a method of reducing adipocytes.

The reduction of the number of adipocytes in localized adiposities occurs by a controlled lipolysis. The pharmaceutical composition induces a direct adipocyte membrane damage and cytoplasm leakage when injected directly into the adipose tissue soon after mixture of the two water solutions (1) and (2), as it generates free oxygen radicals.

Therefore the lipolysis is not mediated by any mechanism of cell apoptosis.

According to a further preferred embodiment the pharmaceutical composition for use according to the present invention is injected into adiposities.

Preferably, said adiposities are localized.

Preferably said pharmaceutical composition is administered to a human.

According to a preferred embodiment in the pharmaceutical composition for use according to the present invention the ferrous iron salt is ferrous gluconate.

Preferably the physiologically acceptable salts of ascorbic acid are selected from sodium ascorbate, calcium ascorbate, potassium ascorbate, magnesium ascorbate or other mineral ascorbates.

According to a further preferred embodiment in the pharmaceutical composition for use according to the present invention water solution (1) has a content in ascorbic acid from 10 to 70% by weight, more preferably from 20 to 60% by weight, most preferably from 30 to 50% by weight.

According to a further preferred embodiment in the pharmaceutical composition for use according to the present invention water solution (2) has a content in ferrous gluconate from 0.01 to 0.1% by weight, more preferably from 0.02 to 0.05% by weight, most preferably from 0.03 to 0.04% by weight.

In a further preferred embodiment, in the pharmaceutical composition for use according to the present invention the water solution (1) has the following composition by weight: sodium ascorbate between 35-45%, disodium EDTA between 0.02-0.1%, water up to 100%.

Preferably said water is purified water.

In a further preferred embodiment in the pharmaceutical composition for use according to the present invention the water solution (2) has the following composition by weight: sucrose between 5-10%, ferrous gluconate between 0.01-0.06%, sodium hydroxide between 0.01-0.06%, hydrochloric acid up to about pH=3.3, purified water up to 100%. In a further preferred embodiment, in the pharmaceutical composition for use according to the present invention, the water solution (1) has the following composition by weight: sodium ascorbate about 40%, disodium EDTA about 0.04%, water up to 100%.

Preferably said water is purified water.

In a further preferred embodiment in the pharmaceutical composition for use according to the present invention the water solution (2) has the following composition by weight: sucrose about 8.47%, ferrous gluconate about 0.037%, sodium hydroxide about 0.035%, hydrochloric acid up to about pH=3.3, purified water up to 100%.

Preferably in the pharmaceutical composition for use according to the present invention solution (1) and solution (2) are mixed in a weight ratio from 2.5-10:40-50, preferably about 5:45.

In a further preferred embodiment the pharmaceutical composition for use according to the present invention is characterized in that it is injected into the cellulite, abdominal fat, double chin (submental fat), adiposity of arms and legs.

Preferably, the pharmaceutical composition for use according to the present invention is for local injection.

In a further preferred embodiment, the pharmaceutical composition for use according to the present invention is for subcutaneous or intradermal injection.

Preferably said pharmaceutical composition is a solution or a suspension.

Preferably, the pharmaceutical composition for use according to the present invention includes solvents, excipients, acceptable diluents or carriers allowing administration by subcutaneous injection.

In a preferred embodiment the pharmaceutical composition for use according to the present invention is administered to a human undergoing a hypocaloric diet or who has completed a slimming diet.

In a further preferred embodiment the administration of the pharmaceutical composition for use according to the present invention is followed by a physical exercise.

The pharmaceutical compositions of the present invention will be prepared according to conventional techniques, using compatible excipients and pharmaceutically acceptable carriers, and may contain, in combination, other active principles with complementary or, in any case, useful activity.

Preferably said active principles are selected from a medium-duration local anaesthetic agent selected from the group of lidocaine, prilocaine and mepivacaine to reduce the discomfort due to the injection.

Preferably, said pharmaceutical composition is administered to a human undergoing a hypocaloric diet or who has completed a slimming diet.

Preferably the administration of said pharmaceutical composition is followed by a physical exercise.

Examples of these pharmaceutical compositions prepared according to the present invention include solutions and suspensions, suitable for intradermal or subcutaneous injection after mixture of the two water solutions (1) and (2).

A further embodiment of the present invention is the use of a composition comprising (1) a water solution of ascorbic acid or a physiologically acceptable salt thereof and (2) a water solution of a ferrous iron salt in reducing adipocytes, wherein said composition is administered by injection within 2 to 10 minutes after mixing said two water solutions (1) and (2).

Preferably said composition is administered by injection within 3 to 6 minutes after mixing said two water solutions (1) and (2), more preferably 3 minutes.

According to a preferred embodiment in the composition according to the present invention the ferrous iron salt is ferrous gluconate.

Preferably the physiologically acceptable salts of ascorbic acid are selected from sodium ascorbate, calcium ascorbate, potassium ascorbate, magnesium ascorbate or other mineral ascorbates.

According to a further preferred embodiment in the composition according to the present invention water solution (1) has a content in ascorbic acid from 10 to 70% by weight, more preferably from 20 to 60% by weight, most preferably from 30 to 50% by weight.

According to a further preferred embodiment in the composition according to the present invention water solution (2) has a content in ferrous gluconate from 0.01 to 0.1% by weight, more preferably from 0.02 to 0.05% by weight, most preferably from 0.03 to 0.04% by weight.

The compositions and the uses of the present invention will now be more fully described by the following examples. It should, however, be noted that such examples are given by way of illustration and not of limitation.

### Definitions

Unless otherwise defined, all terms of art, notations and other scientific terminology used herein are intended to have the meanings commonly understood by those persons skilled in the art to which this disclosure pertains. In some cases, terms with commonly understood meanings are defined herein for clarity and/or for ready reference; thus, the inclusion of such definitions herein should not be construed to represent a substantial difference over what is generally understood in the art.

The terms “approximately” and “about” herein refers to the range of the experimental error, which may occur in a measurement.

The terms “comprising”, “having”, “including” and “containing” are to be construed open-ended terms (i.e. meaning “including, but not limited to”) and are to be considered as providing support also for terms as “consist essentially of”, “consisting essentially of”, “consist of” or “consisting of”.

The terms “consist essentially of”, “consisting essentially of” are to be construed as semi-closed terms, meaning that no other ingredients which materially affects the basic and novel characteristics of the invention are included (optional excipients may thus be included).

The terms “consists of”, “consisting of” are to be construed as closed terms.

The term “localized adiposity” refers to the accumulation of subcutaneous adipose tissue, placed in definite anatomic areas, building up an alteration of the body silhouette.

### BRIEF DESCRIPTION OF THE FIGURES

FIG. 1 shows the reaction kinetic of Fe(II) concentration as a function of time as a mean of two experiments.

FIG. 2 shows the concentration of Fe(III) as a mean of two experiments.

FIG. 3 shows the concentration of ascorbate as a mean of two experiments.

FIG. 4 shows the theoretic quantities of hydroxide radical OH-generated by the reaction.

FIGS. 5 and 6 represent the viability measured by MTT [3-(4,5-Dimethyl-2-thiazolyl)-2,5-diphenyl-2H-tetrazolium bromide)] or NRU (Neutral Red Uptake) of the cells treated with the sample at two concentrations compared to the untreated cells.

FIG. 7 shows the cell viability compared to the negative control (NC) 60 min post-treatment.

FIG. 8 shows the cell viability compared to the negative control (NC) 7 days post-treatment.

FIGS. 9 and 10 show the data on luminescence (index of apoptosis) on treated and untreated cells.

FIGS. 11 and 12 show the data on fluorescence (index of cell death independent of apoptosis) on treated and untreated cells.

FIG. 13 shows the time course of luminescence (RLU) and of fluorescence (RFU) for the compound of Examples 1 and 2.

FIG. 14 shows the time course of glycerol in presence of lipase or not. Glycerol and non-esterified fatty acids (NEFA) derive from the dissociation of triglycerides and is increased by adding lipase to the medium.

### EXAMPLES

### Example 1

A pharmaceutical composition having the following composition wt./wt. % is prepared: Composition of the water solution 1: sodium ascorbate 40%, disodium EDTA 0.04%, purified water q.s.

Composition of the water solution 2: sucrose 8.47%, ferrous gluconate 0.037%, sodium hydroxide 0.035%, hydrochloric acid up to pH=3.3, water q.s.

### Example 2

The study objective was the evaluation of the reaction kinetics after mixing of two water solutions of Example 1. Further objective was the stoichiometric calculation of the mixture components at the different kinetics time points.

The reaction kinetics were evaluated at the different time points after mixing the two water solutions (3 min, 5 min, 10 min, 20 min, 30 min and 60 min) by UV-Vis spectrophotometry (Fe(II) titration), ICP-OES technique (total Fe assay) and HPLC-UV technique (Sodium Ascorbate titration).

After mixing the two water solutions in a solution 1: solution 2=5:45 ratio, the kinetics of increasing Iron (II) concentration were monitored by means of UV-Vis spectroscopy by generating the complex Fe(II)-1,10-phenanthroline and absorbance reading at 509 nm.

A calibration curve was constructed using the Mohr salt FeSO4(NH4)2SO4·6H2O, while the samples, taken at the predefined times from the mixing operation, were diluted ten times before reading, with a time of formation of the Fe(II) complex of 15 minutes.

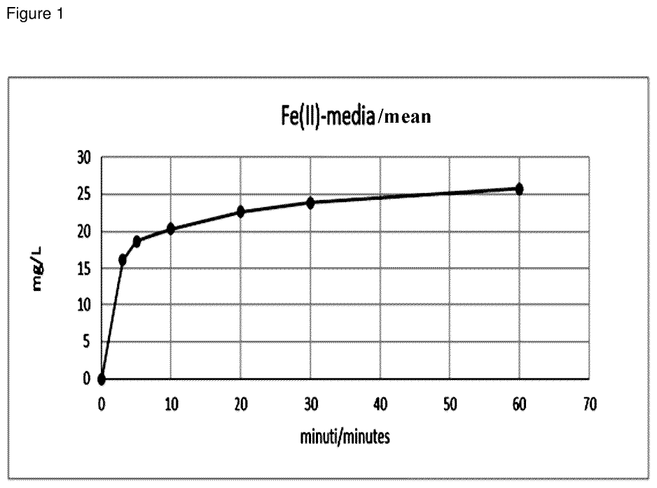

FIG. 1 shows the reaction kinetic of Fe(II) concentration as a function of time, as determined by the colorimetric technique. Data are means of two experiments.

Data from both experiment and average values of the two tests are reported in the following table:

TABLE 1

| Tempo dopo | | | Fe(Il) mg/L - media |
|---|---|---|---|
| miscelazione/ | Fe(II) mg/L - | Fe(II) mg/L - | delle due prove |
| time after mixing | test 1 | test 2 | Mean of two tests |
| 3 min | 16.99 | 15.30 | 16.14 |
| 5 min | 19.70 | 17.72 | 18.71 |
| 10 min | 20.82 | 19.91 | 20.36 |
| 20 min | 23.26 | 22.09 | 22.68 |
| 30 min | 23.96 | 23.72 | 23.84 |
| 60 min | 26.10 | 25.42 | 25.76 |

Table 2 shows the kinetics with the total Iron concentration as a function of time, as determined by the ICP-OES technique. A mean Fe assay among the different withdrawals was calculated, which was found to be fully in accordance with the theoretical expected value (38.70 mg/L).

TABLE 2

| Prelievo/withdrawal | Fe (mg/L) |
|---|---|
| 3 min | 37.57 |
| 5 min | 36.25 |
| 10 min | 38.13 |
| 20 min | 37.14 |
| 30 min | 38.79 |
| 60 min | 40.91 |
| Media/mean | 38.13 |

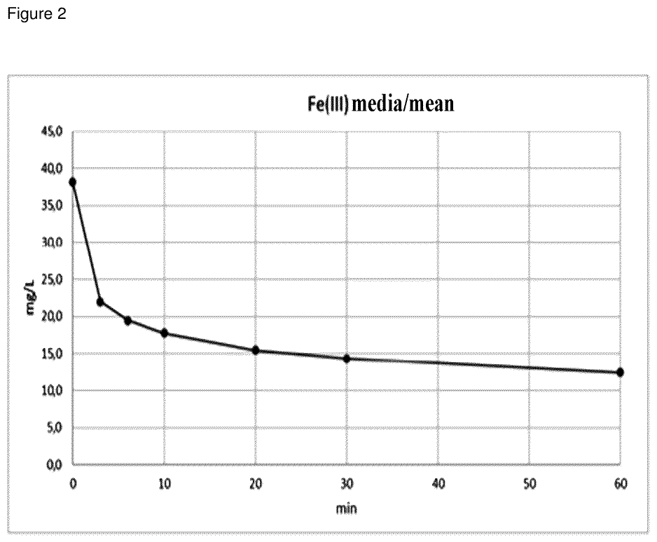

By subtraction it was calculated the concentration of Fe(III) as reported in Table 3 for both experiments and average value, and FIG. 2 for the mean of the two experiments.

TABLE 3

| | Fe(III) | Fe(III) | Fe(III) |
|---|---|---|---|
| Tempo (min) | test 1 | test 2 | media/mean |
| 0 | 38.13 | 38.13 | 38.13 |
| 3 | 21.14 | 22.83 | 21.99 |
| 5 | 18.43 | 20.42 | 19.42 |
| 10 | 17.31 | 18.22 | 17.77 |
| 20 | 14.87 | 16.04 | 15.45 |
| 30 | 14.17 | 14.41 | 14.29 |
| 60 | 12.03 | 12.71 | 12.37 |

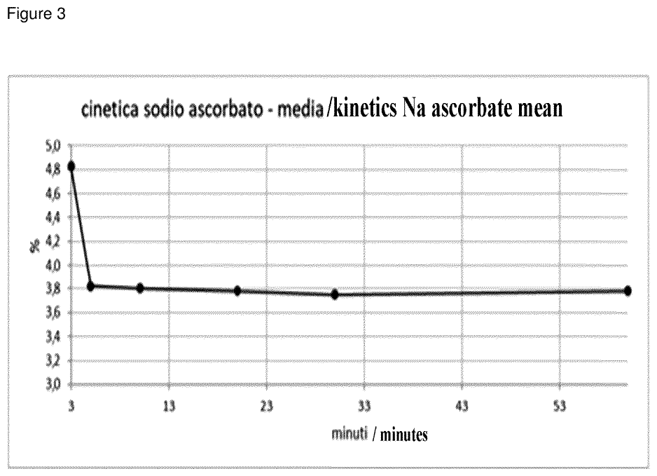

The concentration of ascorbate is reported in Table 4 for both experiments and average value, and in FIG. 3 for the mean of the two experiments.

TABLE 4

| Tempo dopo | Sodio ascorbato/ | Sodio ascorbato/ | Sodio ascorbate/ |
|---|---|---|---|
| miscelazione/ | sodium ascorbate | sodium ascorbate | sodium ascorbate |
| time after | (%) | (%) | (%) |
| mixing | test 1 | test 2 | media/mean |
| 3 min | 4.6 | 5.1 | 4.8 |
| 5 min | 3.8 | 3.9 | 3.8 |
| 10 min | 3.8 | 3.9 | 3.8 |
| 20 min | 3.7 | 3.8 | 3.8 |
| 30 min | 3.7 | 3.8 | 3.7 |
| 60 min | 3.8 | 3.8 | 3.8 |

The concentrations of Fe(II), Fe(III) and ascorbic acid as resulting from the described reaction are summarized in Table 5.

TABLE 5

| | | | Sodio Ascorbato/ |
|---|---|---|---|
| | Fe(II) | Fe(III) | Sodium Ascorbate |
| Tempo/time | (mg/L) | (mg/L) | (%) |
| 3 min | 16.1 | 21.4 | 4.8 |
| 5 min | 18.7 | 17.5 | 3.8 |
| 10 min | 20.4 | 17.8 | 3.8 |
| 20 min | 22.7 | 14.5 | 3.8 |
| 30 min | 23.8 | 15.0 | 3.7 |
| 60 min | 25.8 | 15.1 | 3.8 |

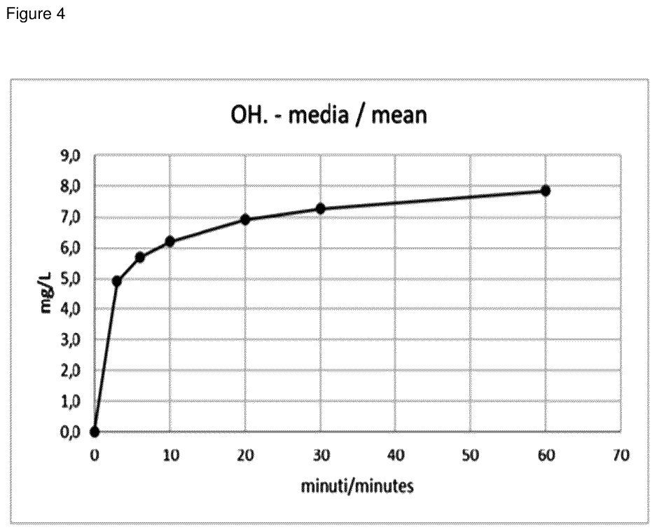

The theoretic quantities of hydroxide radicals generated by the reaction, assuming the stoichiometry according to the described reaction is 1:1 with respect to the amount of Fe(II) generated, are reported in Table 6 and FIG. 4.

TABLE 6

| | | | OH— |
|---|---|---|---|
| | OH— | OH— | (mg/L) |
| Tempo/time | (mg/L) - test 1 | (mg/L) - test 2 | Media/mean |
| 3 min | 5.2 | 4.7 | 4.9 |
| 5 min | 6.0 | 5.4 | 5.7 |
| 10 min | 6.3 | 6.1 | 6.2 |
| 20 min | 7.1 | 6.7 | 6.9 |
| 30 min | 7.3 | 7.2 | 7.3 |
| 60 min | 8.0 | 7.7 | 7.8 |

The kinetic of the involved reaction was characterized by a faster rate in the first 5/10 minutes after mixing the solutions 1 and 2, noticeably slower in the subsequent time points.

### Example 3

Study of the in vitro viability of adipocytes after coming in contact with the composition of Example 1. Several tests have been carried out to assess cell viability kinetics and possible interferences with reagents for the evaluation of viability.

The test has been carried out on human primary pre-adipocytes (ATCC PCS-210-010TM; Batch: 70000726). The cells, seeded in 96-wells plates and differentiated into mature adipocytes, were treated with the sample at two different concentrations (0.75-0.375 ml/ml). The two water solutions of the product were mixed as per Example 2 and after 3 minutes the product was put in contact with the cells. The cells were treated for up to 40 minutes. Having assessed the presence of interference with luminescence signals [relative light unit (RLU)], two other methods were used for the evaluation of viability: MTT [3-(4,5-Dimethyl-2-thiazolyl)-2,5-diphenyl-2H-tetrazolium bromide)] and NRU (Neutral Red Uptake) assay, using separate wells for 4 endpoint times (8-15-20-40 minutes). Cells treated with the medium alone were used as negative control. Wells without cells but treated with the same sample concentrations or negative control were used as an additional control.

For the first endpoint, at the end of the treatment time, the cells were washed but the medium was never completely removed; therefore, a percentage of the sample remained before the addition of the solution with the two dyes. Having observed an effect on the dye, especially with the MTT assay reagent, for the following three time points the sample was completely removed and then two washings were carried out with phosphate buffer saline (PBS) before the addition of the reagents for the evaluation of cell viability.

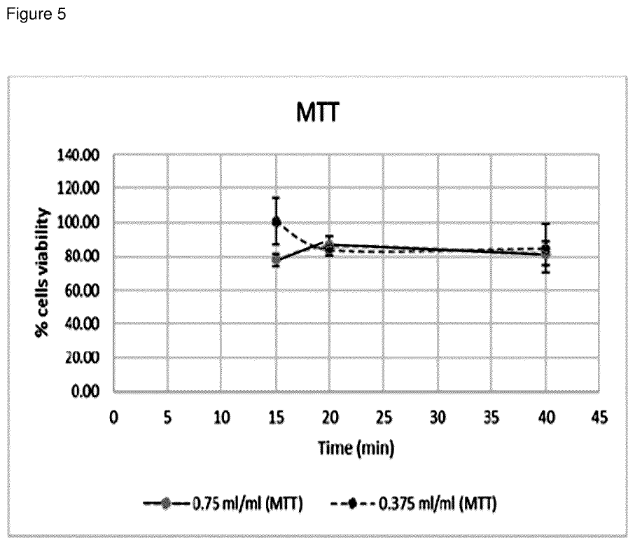

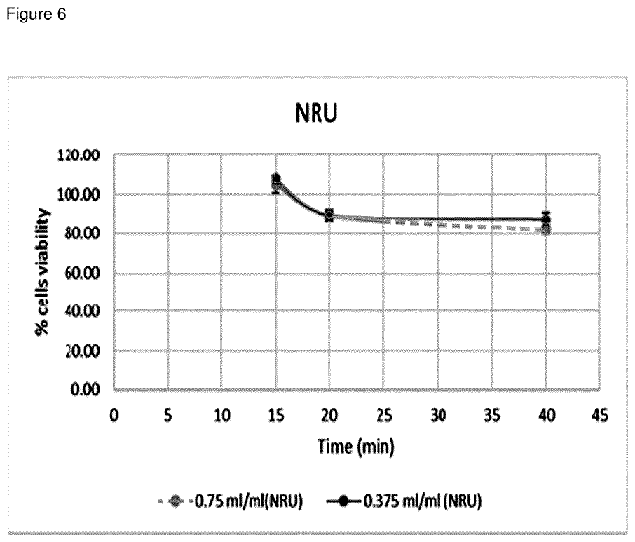

FIGS. 5 and 6 represent the viability of the cells treated with the sample compared to the untreated cells.

The first endpoint (8 min) was excluded because the incomplete removal of the sample lead to an interference with these two reagents and therefore the values were not reliable. With the two assays (MTT or NRU) the result at the first reported endpoint (15′) is slightly different for the higher tested concentration (with MTT assay the sample seems to already result in a mortality effect not yet visible with NRU assay), but at subsequent endpoints results overlap. In general, mortality occurs immediately in the first minutes of contact (within the first 20′) with a value between 10-15% and increases slightly to 18-20% in the following minutes (40′).

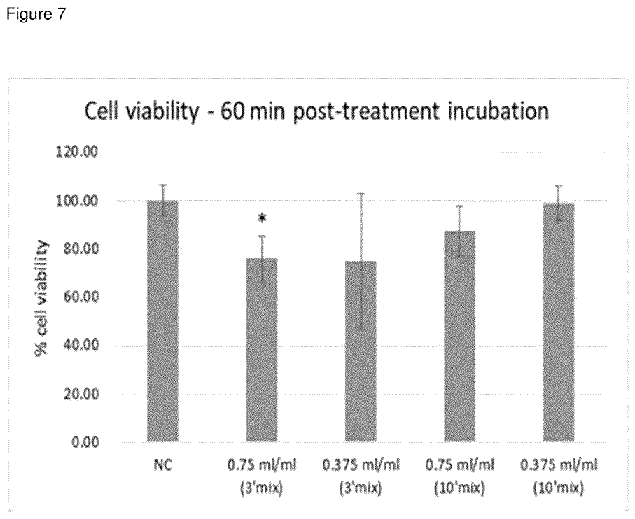

In a further experiment the cells, seeded in 96-wells plates and differentiated into mature adipocytes, were treated with the sample at two different concentrations (0.75-0.375 ml/ml). The two water solutions of the product were mixed according to customer's specifications and after 3 or 10 minutes the product was put in contact with the cells. The cells were treated for up to 40 minutes. In this experiment, the cell viability was not immediately evaluated but at the end of the treatment the cells were washed and fresh culture medium was added for their maintenance. After 1 h and after 7 days the viability was evaluated. Cells treated with the medium alone were used as negative control. Wells without cells but treated with the same sample concentrations or negative control were used as an additional control. The cell viability compared to the negative control 60 min post-treatment is displayed in Table 7 and in FIG. 7.

TABLE 7

| | | 3′mix | | 10′mix | |
|---|---|---|---|---|---|
| | | 0.75 | 0.375 | 0.75 | 0.375 |
| | NC | ml/ml | ml/ml | ml/ml | ml/ml |
| % vitalità/viability | 102.92 | 70.24 | 62.25 | 76.92 | 93.23 |
| | 104.31 | 70.70 | 55.33 | 87.07 | 96.00 |
| | 92.77 | 86.85 | 107.02 | 97.69 | 107.08 |
| media/mean | 100.00 | 75.93 | 74.87 | 87.22 | 98.77 |
| dev.st | 6.30 | 9.46 | 28.06 | 10.39 | 7.33 |
| % mortalità/ | | 24.07 | 25.13 | 12.78 | 1.23 |
| mortality | | | | | |

NC = negative control;
* p < 0.05 vs NC

After 60 minutes of post-treatment incubation, a mortality of 24% is found at the highest concentration tested and when the sample is put in contact with the cells 3 minutes after mixing. The effect is lower (and not statistically significant) if the product is placed in contact 10 minutes after mixing. This difference confirms that the cytotoxic effect is due to the formation of free radicals, the presence of which is maximum after about 3 minutes and later, as expected, the effect is exhausted.

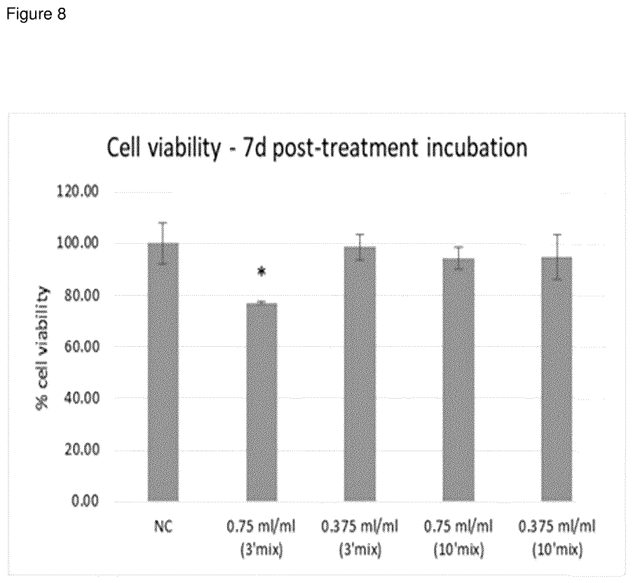

The cell viability compared to the negative control 7 days post-treatment is displayed in Table 8 and FIG. 8.

TABLE 8

| | | 3′mix | | 10′mix | |
|---|---|---|---|---|---|
| | | 0.75 | 0.375 | 0.75 | 0.375 |
| | NC | ml/ml | ml/ml | ml/ml | ml/ml |
| % vitalità/viability | 109.20 | 77.29 | 94.19 | 91.47 | 87.31 |
| | 95.79 | 76.67 | 97.64 | 97.37 | 92.85 |
| | 95.01 | (outlier | 104.08 | (outlier | 104.30 |
| | | replicate) | | replicate) | |
| media/mean | 100.00 | 76.98 | 98.64 | 94.42 | 94.82 |
| dev.st | 7.98 | 0.44 | 5.02 | 4.17 | 8.67 |
| % mortalità/ | | 23.02 | 1.36 | 5.58 | 5.18 |
| mortality | | | | | |

NC = negative control;
* p < 0.05 vs NC

After 7 days of post-treatment incubation, the viability (mortality of 23% at the higher concentration tested and when the sample is put in contact with the cells 3 minutes after mixing) is almost identical to the viability after 60 min (mortality 24% at concentration 0.75 ml/ml when the sample is put in contact with the cells 3 minutes after mixing), therefore it is concluded that the effect of the device is already exhausted after one hour.

### Conclusion

The cell lysis due to the sample occurs in the short term: it arises immediately in the first minutes of contact (within the first 20′) with a value between 10-15% and it increases slightly until it stabilizes with a value between 18-20% in the following 20 minutes (t 40′).

After 60 minutes of post-treatment incubation, a 24% lysis is observed at the highest concentration tested and when the sample is placed in contact with the cells 3 minutes after mixing. The effect is lower (and not statistically significant) if the product is put in contact 10 minutes after mixing: since the effect is due to the formation of free radicals, the presence of the same is maximum after about 3 minutes after which, as expected, the effect is exhausted).

The viability does not decrease after 7 days, but the ratio of living cells in untreated control to living ones in treated cells wells remains the same, indicating that once the device has taken effect, the other live cells, while they continue to grow, no longer replace the gap.

The best effect is observed when the cells are treated with the sample in which the two water solutions have been mixed for a few minutes (3 minutes) while it is decreased if the sample is added with the composition already mixed from 10 min. This confirms that the production of free radicals is the only mechanism involved in the performance of the tested device.

### Example 4

Assessment of any apoptotic potential of the medical device final solution; description of the in vitro necrotizing effect of the medical device final solution; description of the effect on triglyceride release by the adipocytes being exposed to the medical device final solution.

The study objectives were as follows:
To demonstrate the mechanism of action of the composition of Example 1 on possible apoptosis and/or cell necrosis in adipocytes treated with a lipolytic product.
To evaluate the triglycerides' release in the culture medium after treatment with the lipolytic product.

### Background

In the human body, excess fat accumulates in form of triglycerides in cells that are predisposed for this function, i.e. the adipocytes which are found in the white and brown adipose tissue. The white adipose tissue is present as visceral and subcutaneous fat with deposits that can be more or less spread and plays the role of principal energy reserve of the organism, allowing for the mobilization of the fats during the phases of high demand and during moments of nutritional deprivation. In addition to the regulation of the energetic balance of the body, the factors that are secreted from the white adipose tissue play a key role in the modulation of metabolic processes, insulin sensitivity and immunological responses. The adipose tissue of adult humans maintains the capacity to increase the number of adipocytes, suggesting that the differentiation of preadipocytes can occur at any time in response to nutritional and hormonal stimuli. An increase in white adipose tissue other than hyperplasia of adipocytes may be linked to an increase in their dimension (hypertrophy). The triglycerides inside the adipocytes do indeed form vacuoles which can occupy great part of the cytoplasmic space and thus increase significantly the cell volume.

Triglycerides released from adipocytes are used by muscle lipases that break them down into glycerol and free fatty acids. Glycerol is used in the Krebs cycle, free fatty acids are used to produce energy through beta-oxidation. Substances that have been shown to act on adipogenesis and/or on lipolysis, causing the reduction of lipid content of the adipocyte or the necrosis of the adipocytes themselves, may be used as agents for slimming, strengthening and co-adjuvants in the treatment of localized fat deposits.

### Assay Procedures

The test has been carried out on human primary pre-adipocytes (ATCC PCS-210-010TM; Batch: 70000726). The cells, seeded in 96-wells plates and differentiated into mature adipocytes, were treated with the sample at 0.75 ml/ml concentration. The two water solutions of the product were mixed according to the Example 1 and after 5 minutes the product was put in contact with the cells. The cells were treated for up to 40 minutes. At the end of the exposure, the cells were washed and fresh culture medium was added for their maintenance. After 1 h commercial kits were used to evaluate cell apoptosis and/or necrosis (PROMEGA JA1011) as well as triglycerides' release (PROMEGA J3160) in the culture medium. Cells treated with the medium alone were used as negative control. Wells without cells but treated with the same sample concentrations or negative control were used as an additional control.

Adipocyte Apoptosis and/or Necrosis Evaluation

The RealTime-Glo™ Annexin V Apoptosis and Necrosis Assay is an assay that measures the exposure of phosphatidylserine (PS) on the outer leaflet of the cell membrane during the apoptotic process through a luminescent signal. This assay also includes a cell-impermeant, profluorescent DNA dye, which detects necrosis through a fluorescent signal [Niles A L (2017) Real-Time, Bioluminescent Apoptosis Assay https://www.genengnews.com/magazine/302/real-time-bioluminescent-apoptosis-assay/, Accessed 8 Jan. 2023].

Compounds that induce apoptosis will produce time- and dose-dependent increases in luminescence (Annexin V fusion protein binding to exposed PS), which precede temporal increases in fluorescence due to secondary necrosis (loss of membrane integrity). This kinetic difference in the emergence of the signals is the hallmark of the apoptotic phenotype. Compounds that produce increases in luminescence concurrently with increases in fluorescence are not consistent with apoptosis and involve alternative forms of cell death, such as cytoplasmic membrane's damage by free radicals (extremely reactive) resulting in membrane rupture and leakage of cytoplasmic constituents.

### Results

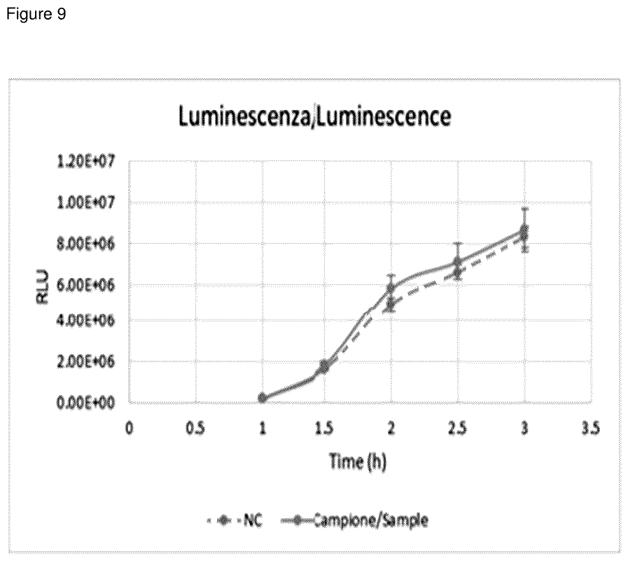

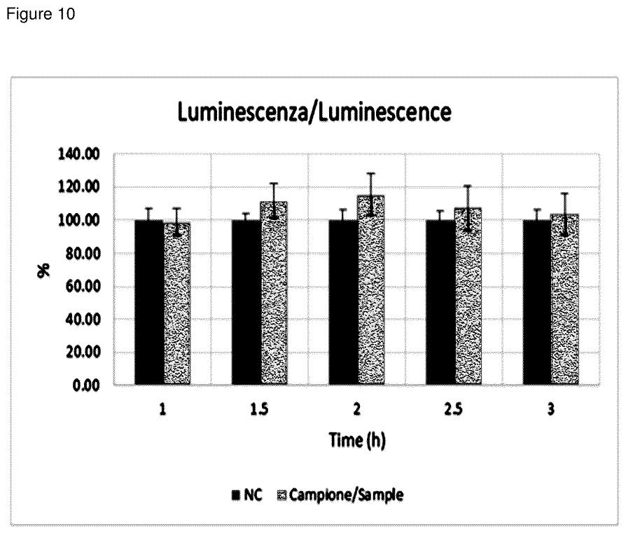

The data on apoptosis are summarized in FIGS. 9 and 10. Luminescence (RLU) is correlated to apoptosis (phosphatidylserine translocation). At the first time point (1 h after the end of treatment) the luminescence values of the treated cells are comparable to the negative control. At the following time points, the values increase but the ratio between sample and control is maintained.

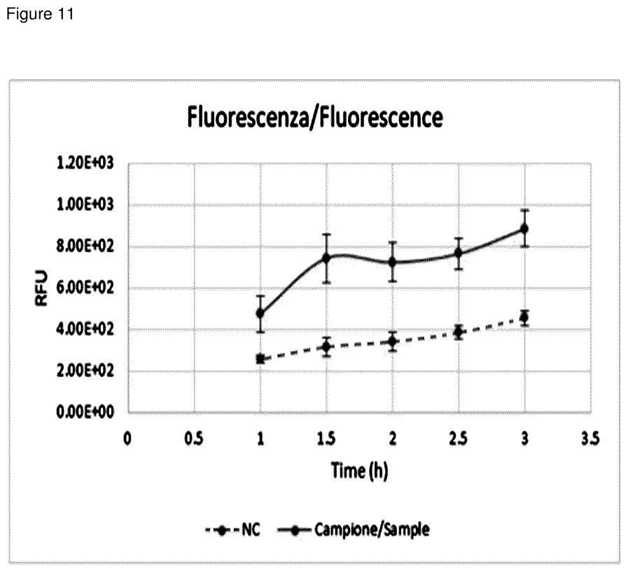

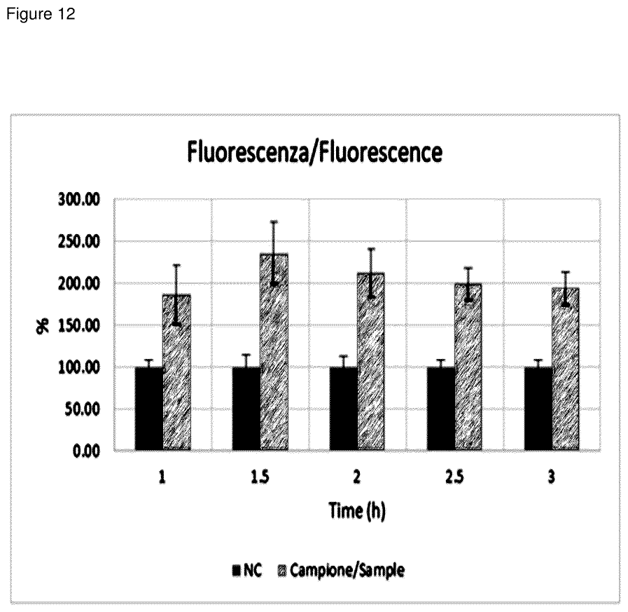

The data on necrosis are summarized in FIGS. 11 and 12. Fluorescence (RFU) is correlated to necrosis (impaired membrane integrity). At the first time point (1 h after the end of treatment) the fluorescence values of the treated cells are significantly higher than the negative control. The values increase up to the end of experiment and the sample maintains a fluorescence value significantly higher than the negative control.

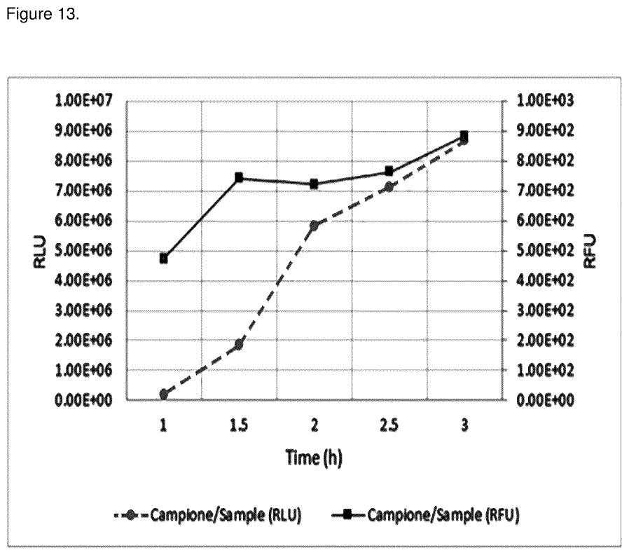

For the test product as per Examples 1 and 2, it is observed that the fluorescence signals (RFU) increase at the same time as the luminescence (RLU) one (see FIG. 13). This pattern confirms that necrosis is not secondary to apoptosis, but it takes place independently. Compounds inducing apoptosis will produce increases in luminescence, which precede temporal increases in fluorescence due to secondary necrosis.

### Effect on Release of Triglycerides

The release of triglycerides was measured by the luminescent method Triglyceride-Glo™ Assay.

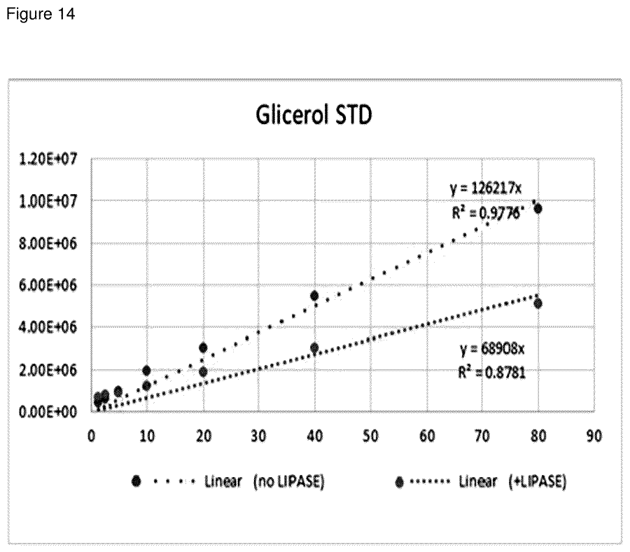

The medium of the treated cells has been collected and the cells, after being washed, have been incubated for 1 h with medium of culture; also this last one has been collected in order to evaluate triglycerides release. These samples were then compared with a glycerol standard plot (see FIG. 14).

The analysis of triglycerides on the medium collected after 1 h from treatment is displayed in Table 9. The release of triglycerides is increased by 30% compared to negative control.

TABLE 9

| | | % rispetto al controllo |
|---|---|---|
| | Trigliceridi/Triglycerides | negativo/compared to |
| | (μM ± stand. Dev.) | negative control |
| Controllo negativo/ | 59.68 ± 1.59 | — |
| Negative control | | |
| Lipolytic product | 77.79 ± 9.36 | 130.35% |
| Phase 1 + Lipolytic | | P value < 0.01 |
| product Phase 2 | | |

### Conclusion

Lipolytic solution as tested in the above conditions:
1. is able to induce cell lysis, whereas it does not induce apoptosis;
2. increases the triglycerides release compared to untreated control cell cultures, suggesting membrane damage and cytoplasmic leakage induced by free radicals developed by the product;
3. the 30% increase in triglycerides correlates well with the 24% decrease in cell viability demonstrated in Example 3 and confirms the hypothesis formulated in the previous point.

1. A pharmaceutical composition comprising (1) a water solution of ascorbic acid or a physiologically acceptable salt thereof and (2) a water solution of a ferrous iron salt for use as a medicament, wherein said composition is administered by injection within 10 minutes after mixing said two water solutions (1) and (2).

2. A pharmaceutical composition according to claim 1, wherein said composition is administered by injection within 2 to 10 minutes after mixing said two water solutions (1) and (2).

3. A pharmaceutical composition according to claim 1 or 2, for use in a method for reducing adipocytes.

4. A pharmaceutical composition for use according to any of the preceding claims, wherein said composition is injected into adiposities.

5. A pharmaceutical composition for use according to any of the preceding claims, wherein said composition is administered to a human.

6. A pharmaceutical composition for use according to any of the preceding claims, wherein the ferrous iron salt is ferrous gluconate.

7. A pharmaceutical composition for use according to any of the preceding claims, wherein physiologically acceptable salts of ascorbic acid are selected from sodium ascorbate, calcium ascorbate, potassium ascorbate, magnesium ascorbate or other mineral ascorbates.

8. A pharmaceutical composition for use according to any of the preceding claims, wherein water solution (1) has a content in ascorbic acid from 10 to 70% by weight, more preferably from 20 to 60% by weight, most preferably from 30 to 50% by weight.

9. A pharmaceutical composition for use according to any of the preceding claims, wherein water solution (2) has a content in ferrous gluconate from 0.01 to 0.1% by weight, more preferably from 0.02 to 0.05% by weight, most preferably from 0.03 to 0.04% by weight.

10. A pharmaceutical composition for use according to any of the preceding claims, wherein water solution (1) has the following composition by weight: sodium ascorbate about 40%, disodium EDTA about 0.04%, water up to 100%.

11. A pharmaceutical composition for use according to any of the preceding claims, wherein water solution (2) has the following composition: sucrose about 8.47% by weight, ferrous gluconate about 0.037% by weight, sodium hydroxide about 0.035% by weight, hydrochloric acid up to about pH=3.3, water up to 100% by weight.

12. A pharmaceutical composition for use according to any of the preceding claims, wherein solution (1) and solution (2) are mixed in a weight ratio from 2.5-10:40-50, preferably about 5:45.

13. A pharmaceutical composition for use according to any of the preceding claims, characterized in that it is injected into the cellulite, the abdominal fat, the double chin (submental fat) and/or the adiposity of arms and legs.

14. A pharmaceutical composition for use according to any of the preceding claims, wherein said composition is for local injection.

15. A pharmaceutical composition for use according to any of the preceding claims, wherein said composition is for subcutaneous or intradermal injection.

16. A pharmaceutical composition for use according to any of the preceding claims, wherein said composition includes solvents, excipients, acceptable diluents or carriers for administration by subcutaneous injection.

17. A pharmaceutical composition for use according to any of the preceding claims, wherein said composition includes a medium-duration local anaesthetic agent from the group of lidocaine, prilocaine or mepivacaine.

18. A pharmaceutical composition for use according to any of the preceding claims, wherein said composition is administered to a human undergoing a hypocaloric diet or who has completed a slimming diet.

19. A pharmaceutical composition for use according to any of the preceding claims, wherein administration of said composition is followed by a physical exercise.
